SEQUENCE LISTING (2 sequences)

SEQ ID NO: 1 - protein, 969 aa, Homo sapiens
MPPRAPPAPGPRPPPRAAAATDTAAGAGGAGGAGGAGGPGFRPLAPRPWRWLLLLALPAACSAPPPRPVYTNHWAVQVLGGPAEADRVAAAHGYLNLGQIGNLEDYYHFYHSKTFKRSTLSSRGPHTFLRMDPQVKWLQQQEVKRRVKRQVRSDPQALYFNDPIWSNMWYLHCGDKNSRCRSEMNVQAAWKRGYTGKNVVVTILDDGIERNHPDLAPNYDSYASYDVNGNDYDPSPRYDASNENKHGTRCAGEVAASANNSYCIVGIAYNAKIGGIRMLDGDVTDVVEAKSLGIRPNYIDIYSASWGPDDDGKTVDGPGRLAKQAFEYGIKKGRQGLGSIFVWASGNGGREGDYCSCDGYTNSIYTISVSSATENGYKPWYLEECASTLATTYSSGAFYERKIVTTDLRQRCTDGHTGTSVSAPMVAGIIALALEANSQLTWRDVQHLLVKTSRPAHLKASDWKVNGAGHKVSHFYGFGLVDAEALVVEAKKWTAVPSQHMCVAASDKRPRSIPLVQVLRTTALTSACAEHSDQRVVYLEHVVVRTSISHPRRGDLQIYLVSPSGTKSQLLAKRLLDLSNEGFTNWEFMTVHCWGEKAEGQWTLEIQDLPSQVRNPEKQGKLKEWSLILYGTAEHPYHTFSAHQSRSRMLELSAPELEPPKAALSPSQVEVPEDEEDYTAQSTPGSANILQTSVCHPECGDKGCDGPNADQCLNCVHFSLGSVKTSRKCVSVCPLGYFGDTAARRCRRCHKGCETCSSRAATQCLSCRRGFYHHQEMNTCVTLCPAGFYADESQKNCLKCHPSCKKCVDEPEKCTVCKEGFSLARGSCIPDCEPGTYFDSELIRCGECHHTCGTCVGPGREECIHCAKNFHFHDWKCVPACGEGFYPEEMPGLPHKVCRRCDENCLSCAGSSRNCSRCKTGFTQLGTSCITNHTCSNADETFCEMVKSNRLCERKLFIQFCCRTCLLAG

SEQ ID NO: 2 - protein, 13 aa, synthetic construct
NVVVTILDDGIER

(19) United States
(12) Patent Application Publication
(10) Pub. No.: US 2024/0353404 A1
(43) Pub. Date: Oct. 24, 2024

(54) METHODS AND MATERIALS FOR IDENTIFYING AND TREATING MEMBRANOUS NEPHROPATHY

(71) Applicant: Mayo Foundation for Medical Education and Research, Rochester, MN (US)

(72) Inventors: Sanjeev Sethi, Rochester, MN (US); Fernando C. Fervenza, Rochester, MN (US); Benjamin J. Madden, Stewartville, MN (US)

(73) Assignee: Mayo Foundation for Medical Education and Research, Rochester, MN (US)

(21) Appl. No.: 18/634,194

(22) Filed: Apr. 12, 2024

Related U.S. Application Data
(60) Provisional application No. 63/461,494, filed on Apr. 24, 2023.

(51) Int. Cl. G01N 33/564; A61K 38/13; A61K 39/00; A61P 13/12; C07K 16/28

(52) U.S. Cl. CPC G01N 33/564; A61K 38/13; A61P 13/12; C07K 16/2887; A61K 2039/505; G01N 2333/96411; G01N 2800/347

(57) ABSTRACT

This document relates to methods and materials involved in identifying and/or treating mammals having membranous nephropathy (e.g., membranous nephropathy with an elevated level of a proprotein convertase subtilisin/kexin type 6 (PCSK6) polypeptide in kidney tissue such as the glomerular basement membrane (GBM)). For example, methods and materials for administering one or more immunosuppressive agents to treat a mammal (e.g., a human) identified as having membranous nephropathy are provided.

### CROSS-REFERENCE TO RELATED APPLICATIONS

This application claims the benefit of U.S. Patent Application Ser. No. 63/461,494, filed on Apr. 24, 2023. The disclosure of the prior application is considered part of, and is incorporated by reference in, the disclosure of this application.

### SEQUENCE LISTING

This application contains a Sequence Listing that has been submitted electronically as an XML file named “07039-2209001 SL.xml.” The XML file, created on Mar. 12, 2024, is 4000 bytes in size. The material in the XML file is hereby incorporated by reference in its entirety.

### TECHNICAL FIELD

This document relates to methods and materials involved in identifying and/or treating mammals having membranous nephropathy (e.g., membranous nephropathy with an elevated level of a proprotein convertase subtilisin/kexin type 6 (PCSK6) polypeptide in kidney tissue such as the glomerular basement membrane (GBM)). For example, this document provides methods and materials for administering one or more immunosuppressive agents to treat a mammal (e.g., a human) having membranous nephropathy.

### BACKGROUND

Membranous nephropathy results from subepithelial deposition of immune complexes along the GBM. Membranous nephropathy is often classified into primary membranous nephropathy, where there is no identifiable underlying disease association, and secondary membranous nephropathy, where membranous nephropathy may be associated with an autoimmune disease, infection, malignancy, hematopoietic stem cell transplant, etc. (Beck et al., J. Clin. Invest., 124:2307-2314 (2014); Ronco et al., The Lancet, 385:1983-1992 (2015); and Couser, Clin. J. Am. Soc. Nephrol., 12:983-997 (2017)). Target antigens for many types of membranous nephropathy have been identified (Beck et al., New Eng. J. Med., 361:11-21 (2009); Tomas et al., New Eng. J. Med, 371:2277-2287 (2014); Sethi, J. Am. Soc. Nephrol., 32:268 (2021); Sethi et al., J. Am. Soc. Nephrol, 30:1123-1136 (2019); Sethi et al., Kidney Int., 97:163-174 (2020); Sethi et al., Kidney Int., 98:1253-1265 (2020); Sethi et al., J. Am. Soc. Nephrol., 32:1249-1261 (2021); Caza et al., Kidney Int., 100:171-181 (2020); and Al-Rabadi et al., J. Am. Soc. Nephrol., 32:1666-1681 (2021)).

Chronic use of non-steroidal anti-inflammatory drugs (NSAIDs) is associated with analgesic nephropathy characterized by a chronic interstitial nephritis (De Broe et al., New Engl. J. Med., 338:446-452 (1998)) and with the development of nephrotic syndrome and membranous nephropathy (Clive et al., N. Engl. J. Med., 310:563-572 (1984); Radford Jr. et al., JAMA, 276:466-469 (1996); Mérida et al., Clin. J. Am. Soc. Nephrol., 14:1280-1282 (2019); Nawaz et al., Am. J. Kidney Dis., 62:1012-1017 (2013); Bakhriansyah et al., Clin. J. Am. Soc. Nephrol., 14:1355 (2019); and Tattersall et al., Postgrad. Med. J., 68:392 (1992)). The target antigen(s) in NSAID-associated membranous nephropathy is unknown.

### SUMMARY

This document provides methods and materials involved in identifying and/or treating mammals (e.g., humans) having membranous nephropathy (e.g., membranous nephropathy with an elevated level of a PCSK6 polypeptide in kidney tissue such as the GBM). For example, this document provides methods and materials for identifying a mammal (e.g., a human) having membranous nephropathy as having an elevated level of a PCSK6 polypeptide in kidney tissue such as the GBM that can serve as a target antigen in membranous nephropathy. This document also provides methods and materials for identifying a mammal (e.g., a human) having membranous nephropathy as having the presence of autoantibodies having binding specificity for a PCSK6 polypeptide. As described herein, mammals (e.g., humans) having membranous nephropathy can be identified as having an elevated level of a PCSK6 polypeptide in kidney tissue such as the GBM. In such cases, the mammal can be classified as having a form of membranous nephropathy that includes an elevated level of a PCSK6 polypeptide in kidney tissue such as the GBM. As also described herein, mammals (e.g., humans) having membranous nephropathy can be identified as having autoantibodies having binding specificity for a PCSK6 polypeptide. In such cases, the mammal can be classified as having a form of membranous nephropathy that includes the presence of autoantibodies having binding specificity for a PCSK6 polypeptide. Identifying mammals (e.g., humans) as having membranous nephropathy that includes an elevated level of a PCSK6 polypeptide in kidney tissue such as the GBM and/or that includes the presence of autoantibodies having binding specificity for a PCSK6 polypeptide can allow clinicians and patients to proceed with appropriate membranous nephropathy treatment options.

This document also provides methods and materials for treating membranous nephropathy. For example, a mammal (e.g., a human) having membranous nephropathy that was identified as having an elevated level of a PCSK6 polypeptide in kidney tissue such as the GBM, as having autoantibodies having binding specificity for a PCSK6 polypeptide, or as having both an elevated level of a PCSK6 polypeptide in kidney tissue such as the GBM and autoantibodies having binding specificity for a PCSK6 polypeptide can be administered one or more immunosuppressive agents (e.g., one or more corticosteroids, cyclosporine, and/or one or more B-cell reduction or depletion agents such as rituximab) to reduce inflammation and/or B-cell autoantibody production. As described herein, mammals (e.g., humans) having membranous nephropathy and identified as having an elevated level of a PCSK6 polypeptide in kidney tissue such as the GBM and/or as having autoantibodies having binding specificity for a PCSK6 polypeptide have a form of membranous nephropathy that is caused by the presence of antigen-autoantibody complexes where the antigen is a PCSK6 polypeptide. In such cases, the mammal (e.g., human) can be effectively treated using one or more immunosuppressive agents (e.g., one or more corticosteroids, cyclosporine, and/or one or more B-cell reduction or depletion agents such as rituximab) to reduce inflammation and/or B-cell autoantibody production. Having the ability to administer one or more immunosuppressive agents to mammals (e.g., humans) (a) having membranous nephropathy and (b) identified as having an elevated level of a PCSK6 polypeptide in kidney tissue such as the GBM and/or as having autoantibodies having binding specificity for a PCSK6 polypeptide can allow clinicians and patients to treat membranous nephropathy effectively.

In some cases, identification of the target antigen and autoantibodies can be used to diagnosis and/or to manage treatment of a mammal (e.g., a human) with membranous nephropathy. For example, a mammal (e.g., a human) having membranous nephropathy (e.g., membranous nephropathy having an elevated level of a PCSK6 polypeptide in kidney tissue such as the GBM and/or having autoantibodies having binding specificity for a PCSK6 polypeptide) can be administered one or more immunosuppressive agents (e.g., one or more corticosteroids, cyclosporine, and/or one or more B-cell reduction or depletion agents such as rituximab) to treat membranous nephropathy. In some cases, the response to the immunosuppressive treatment can be monitored for a decrease or complete elimination of the autoantibodies having binding specificity for a PCSK6 polypeptide. In some cases, the response to treatment can be monitored by examining a kidney biopsy for a decrease or elimination of a PCSK6 polypeptide. In some cases, a mammal (e.g., a human) having membranous nephropathy can be administered one or more immunosuppressive agents (e.g., one or more corticosteroids, cyclosporine, and/or one or more B-cell reduction or depletion agents such as rituximab) to treat membranous nephropathy based on the presence of an autoantibody having binding specificity for a PCSK6 polypeptide in the absence of evaluating a kidney biopsy for an elevated level of a PCSK6 polypeptide. Although kidney biopsies showing an accumulation of a PCSK6 polypeptide in kidney tissue such as the GBM may be considered an effective manner for diagnosis of membranous nephropathy, the presence of autoantibodies having binding specificity for a PCSK6 polypeptide can be used to identify membranous nephropathy associated with accumulation of PCSK6 polypeptides without the need for performing a kidney biopsy.

Also, having the ability to identify a mammal as having membranous nephropathy based, at least in part, on (a) having an elevated level of a PCSK6 polypeptide in kidney tissue such as the GBM and/or (b) having autoantibodies having binding specificity for a PCSK6 polypeptide provides a unique and unrealized opportunity to provide a safe and effective use of one or more immunosuppressive agents (e.g., one or more corticosteroids, cyclosporine, and/or one or more B-cell reduction or depletion agents such as rituximab) to treat membranous nephropathy in this identified patient population.

In general, one aspect of this document features methods for identifying a mammal having been administered as having membranous nephropathy comprising an elevated level of a PCSK6 polypeptide within kidney tissue of said mammal. The methods can include, or consist essentially of, (a) determining the presence or absence of autoantibodies specific for said PCSK6 polypeptide within a sample obtained from said mammal, (b) classifying said mammal as having said membranous nephropathy if said autoantibodies are present within said sample, and (c) classifying said mammal as not having said membranous nephropathy if said autoantibodies are absent within said sample. The mammal can be a human. The mammal can be a mammal that was previously administered a NSAID. The sample can be a blood sample. The membranous nephropathy can lack, within said kidney tissue, an elevated level of a protocadherin-7 (PCDH7) polypeptide, a semaphorin-3B (SEMA3B) polypeptide, a neural epidermal growth factor (EGF)-like 1 (NELL-1) polypeptide, an exostosin 1 (EXT1) polypeptide, an exostosin 2 (EXT2) polypeptide, a PLA2R polypeptide, a THSD7A polypeptide, a protocadherin FAT1 (FAT1) polypeptide, or a neuron-derived neurotrophic factor (NDNF) polypeptide. The membranous nephropathy can lack, within said kidney tissue, an elevated level of a PCDH7 polypeptide, a SEMA3B polypeptide, a NELL-1 polypeptide, an EXT1 polypeptide, an EXT2 polypeptide, a PLA2R polypeptide, a THSD7A polypeptide, a FAT1 polypeptide, and a NDNF polypeptide. The method can include determining the presence of said autoantibodies within said sample and classifying said mammal as having said membranous nephropathy. The method can include determining the absence of said autoantibodies within said sample and classifying said mammal as not having said membranous nephropathy.

In another aspect, this document features methods for identifying a mammal as having a membranous nephropathy having kidney tissue comprising an elevated level of a PCSK6 polypeptide. The methods can include, or consist essentially of, (a) determining the presence or absence of said kidney tissue within a sample obtained from said mammal, (b) classifying said mammal as having said membranous nephropathy if said presence is determined, and (c) classifying said mammal as not having said membranous nephropathy if said absence is determined. The mammal can be a human. The mammal can be a mammal that was previously administered a NSAID. The kidney tissue can lack an elevated level of a PCDH7 polypeptide, a SEMA3B polypeptide, a NELL-1 polypeptide, an EXT1 polypeptide, an EXT2 polypeptide, a PLA2R polypeptide, a THSD7A polypeptide, a FAT1 polypeptide, or a NDNF polypeptide. The kidney tissue can lack an elevated level of a PCDH7 polypeptide, a SEMA3B polypeptide, a NELL-1 polypeptide, an EXT1 polypeptide, an EXT2 polypeptide, a PLA2R polypeptide, a THSD7A polypeptide, a FAT1 polypeptide, and a NDNF polypeptide. The method can include determining said presence and classifying said mammal as having said membranous nephropathy. The method can include determining said absence and classifying said mammal as not having said membranous nephropathy.

In another aspect, this document features methods for identifying a mammal as having a membranous nephropathy having autoantibodies specific for a PCSK6 polypeptide. The methods can include, or consist essentially of, (a) determining the presence or absence of said autoantibodies within a sample obtained from said mammal, (b) classifying said mammal as having said membranous nephropathy if said autoantibodies are present within said sample, and (c) classifying said mammal as not having said membranous nephropathy if said autoantibodies are absent within said sample. The mammal can be a human. The mammal can be a mammal that was previously administered a NSAID. The sample can be a blood sample. The kidney tissue of said mammal can lack an elevated level of a PCDH7 polypeptide, a SEMA3B polypeptide, a NELL-1 polypeptide, an EXT1 polypeptide, an EXT2 polypeptide, a PLA2R polypeptide, a THSD7A polypeptide, a FAT1 polypeptide, or a NDNF polypeptide. The kidney tissue of said mammal can lack an elevated level of a PCDH7 polypeptide, a SEMA3B polypeptide, a NELL-1 polypeptide, an EXT1 polypeptide, an EXT2 polypeptide, a PLA2R polypeptide, a THSD7A polypeptide, a FAT1 polypeptide, and a NDNF polypeptide. The method can include determining said presence within said sample and classifying said mammal as having said membranous nephropathy. The method can include determining said absence within said sample and classifying said mammal as not having said membranous nephropathy.

In another aspect, this document features methods for treating membranous nephropathy where the methods can include, or consist essentially of, (a) identifying a mammal as having membranous nephropathy comprising (i) autoantibodies specific for a PCSK6 polypeptide, (ii) kidney tissue comprising an elevated level of said PCSK6 polypeptide, or both (i) and (ii), and (b) administering an immunosuppressant to said mammal. The mammal can be a human. The mammal can be a mammal that was previously administered a NSAID. The mammal can be identified as having said membranous nephropathy comprising said autoantibodies. The mammal can be identified as having said membranous nephropathy comprising said kidney tissue. The immunosuppressant can be a B-cell inhibitor. The immunosuppressant can be a calcineurin inhibitor. The immunosuppressant can be an mTOR inhibitor. The immunosuppressant can be rituximab, cyclosporine, tacrolimus, sirolimus, or everolimus. The level of autoantibodies present within said mammal can be reduced by at least 5 percent following said administering step. The level of autoantibodies present within said mammal can be reduced by at least 25 percent following said administering step. The level of autoantibodies present within said mammal can be reduced by at least 50 percent following said administering step.

In another aspect, this document features methods for treating membranous nephropathy where the methods can include, or consist essentially of, administering an immunosuppressant to a mammal identified as having membranous nephropathy comprising (i) autoantibodies specific for a PCSK6 polypeptide, (ii) kidney tissue comprising an elevated level of said PCSK6 polypeptide, or both (i) and (ii). The mammal can be a human. The mammal can be a mammal that was previously administered a NSAID. The mammal can have been identified as having said membranous nephropathy comprising said autoantibodies. The mammal can have been identified as having said membranous nephropathy comprising said kidney tissue. The immunosuppressant can be a B-cell inhibitor. The immunosuppressant can be a calcineurin inhibitor. The immunosuppressant can be an mTOR inhibitor. The immunosuppressant can be rituximab, cyclosporine, tacrolimus, sirolimus, or everolimus. The level of autoantibodies present within said mammal can be reduced by at least 5 percent following said administering step. The level of autoantibodies present within said mammal can be reduced by at least 25 percent following said administering step. The level of autoantibodies present within said mammal can be reduced by at least 50 percent following said administering step.

In another aspect, this document features methods for treating a mammal having membranous nephropathy and kidney tissue comprising an elevated level of a PCSK6 polypeptide where the methods can include, or consist essentially of, administering an immunosuppressant to said mammal. The mammal can be a human. The mammal can be a mammal that was previously administered a NSAID. The mammal can have autoantibodies specific for said PCSK6 polypeptide. The mammal can have been identified as having said kidney tissue. The immunosuppressant can be a B-cell inhibitor. The immunosuppressant can be a calcineurin inhibitor. The immunosuppressant can be an mTOR inhibitor. The immunosuppressant can be rituximab, cyclosporine, tacrolimus, sirolimus, or everolimus. The level of autoantibodies present within said mammal can be reduced by at least 5 percent following said administering step. The level of autoantibodies present within said mammal can be reduced by at least 25 percent following said administering step. The level of autoantibodies present within said mammal can be reduced by at least 50 percent following said administering step.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1. Discovery and validation cohorts: Initial discovery cohort of 250 cases of PLA2R-negative membranous nephropathy (MN) revealed five cases of PCSK6-associated MN. All five cases had history of prolonged use of NSAIDs. In the validation cohort of eight cases of PCSK6-associated MN there was history of NSAID use in 5 cases, in 1 case NSAID use was not known, and in the remaining 2 cases there was no history of NSAID use.

FIGS. 2A-D. Proteomic identification of PCSK6 in PLA2R-negative MN. FIGS. 2A-2C: Discovery cohort. Glomeruli were microdissected and analyzed using mass spectrometry. FIG. 2A) Detection of PCSK6 in 5 cases of PLA2R-negative MN (top row). Numbers in boxes represent total spectral counts of MS/MS matches to a respective protein. All five biopsies show high total spectral counts for PCSK6 and IgG1, IgG2, IgG3 subtypes, and baseline spectral counts of PLA2R were also detected in 1 of the 5 cases. For comparison, the pooled total spectral counts from six control cases (time 0 protocol transplant biopsies) are also shown, PCSK6 was not present in the control cases. FIG. 2B) Representative sequence coverage map of PCSK6 from one case (SEQ ID NO:1). Amino acids highlighted in bold letters over shaded background are the amino acids detected. FIG. 2C) The MS/MS spectra figure from Scaffold viewer of ion 721.90 [M+2H]²⁺ highlighting the detected b-ions and y-ions matching the theoretical fragment masses listed in the table for the PCSK6 peptide NVVVTILDDGIER (SEQ ID NO:2). FIG. 2D) Validation cohort. Volcano plot showing PCSK6 enriched within protein G immunoprecipitated with the highest fold change compared to other cases of MN of known types (PLA2R, THSD7A, NELL1, EXT2) and other ‘quadruple negative’ MN cases.

FIGS. 3A-3D. Immunohistochemical (IHC) staining, immunofluorescence and confocal IF staining for PCSK6. FIG. 3A). Discovery cohort. IHC staining showing granular staining for PCSK6 along GBM in all five cases. A control PLA2R-positive is negative for PCSK6 along the GBM. FIG. 3B) Validation cohort. Immunofluorescence microscopy showing granular staining for PCSK6 along the GBM in six cases. The images are at 400× magnification, scale bar=20 μm. The remaining cases in the validation cohort (not shown) were validated by laser capture microdissection. FIG. 3C) Validation cohort. Confocal microscopy of a representative case of PCSK6-associated MN showing glomeruli double labeled with anti-human IgG and anti-PCSK6, along with the merged image. Top panel is a PCSK6-associated MN from the validation cohort. Bottom panel is a PLA2R-associated MN showing only staining for IgG and no staining for PCSK6. The images are at 400× magnification, scale bar=20 μm. FIG. 3D) IgG subtypes done in 3 cases of PCSK6-associated MN from the validation cohort showing co-dominant staining of IgG1 and IgG4 in both cases, with negative staining for IgG2 and IgG3. The images are at 400× magnification, scale bar=20 μm.

FIGS. 4A-4B. FIG. 4A) Western Blot analysis showing IgG from eluate of PCSK6-associated MN bind to reduced PCSK6 (1400 ng loaded in each lane). Control (lane 1): Reduced PCSK6 is detected by mouse anti-human PCSK6 (1:15000) at approximately 37 kDa (arrow). Eluate (lane 2): reduced PCSK6 (arrow) is detected using eluate from PCSK6-associated MN using a secondary anti-human IgG (1:5000). The binding is not detected using eluate from PLA2R-associated MN (lane 3). Near infrared fluorescence was detected at the 700 and 800 nm channel and by chemiluminescence in the Odyssey Infrared Imaging System (LI-COR® Biosciences, Lincoln, NE). The reading was done for 10 minutes in both channels (total 30 min). FIG. 4B) Western Blot analysis showing IgG patient serum of PCSK6-associated MN bind to reduced PCSK6 (1600 ng loaded in each lane). Patient serum: anti-PCSK6 IgG from patient serum (1:50) is detected using anti-human IgG (1:5000) bound to PCSK6 in between 37-50 kDa, at baseline (proteinuria 6.0 mg/dL/24 hours) (1) and after treatment with rituximab (RTX) (18 months, proteinuria 0.5 mg/dL/24 hours) (2). Anti-PCSK6 IgG are not detected using serum from PLA2R-associated MN (3) and MPO-AAV-GN (4). Near infrared fluorescence was detected at the 700 and 800 nm channel and by chemiluminescence in the Odyssey Infrared Imaging System (LI-CORR Biosciences, Lincoln, NE). The reading was done for 10 minutes in both channels (total 30 minutes).

### DETAILED DESCRIPTION

This document provides methods and materials for identifying and/or treating mammals (e.g., humans) having membranous nephropathy (e.g., membranous nephropathy with an elevated level of a PCSK6 polypeptide in kidney tissue such as the GBM). For example, this document provides methods and materials for identifying a mammal (e.g., a human) having membranous nephropathy as having (a) autoantibodies specific for a PCSK6 polypeptide and/or (b) kidney tissue such as the GBM having an elevated level of a PCSK6 polypeptide.

Any appropriate mammal having membranous nephropathy can be identified as having (a) autoantibodies specific for a PCSK6 polypeptide and/or (b) kidney tissue (e.g., the GBM) having an elevated level of a PCSK6 polypeptide. Examples of mammals having membranous nephropathy that can be identified as having (a) autoantibodies specific for a PCSK6 polypeptide and/or (b) kidney tissue (e.g., the GBM) having an elevated level of a PCSK6 polypeptide as described herein include, without limitation, humans, non-human primates (e.g., monkeys), dogs, cats, horses, cows, pigs, sheep, rabbits, mice, and rats. For example, humans having membranous nephropathy can be identified as having (a) autoantibodies specific for a PCSK6 polypeptide and/or (b) kidney tissue such as the GBM having an elevated level of a PCSK6 polypeptide as described herein.

In some cases, a mammal having membranous nephropathy also can be a mammal that previously was administered or that previously was self-administering one or more NSAIDs for extended periods of time (e.g., longer than three months of daily or at least weekly use). For example, a mammal having membranous nephropathy can be a chronic NSAIDs user. In some cases, chronic NSAID use can include taking one or more NSAIDs for three or more (e.g., three, four, five, six, seven, or more) times a week. In some cases, chronic NSAID use can include taking one or more NSAIDs for three or more (e.g., three, four, five, six, seven, or more) months. In some cases, chronic NSAID use can include taking one or more NSAIDs for three or more (e.g., three, four, five, six, seven, or more) times a week for three or more (e.g., three, four, five, six, seven, or more) months. For example, chronic NSAID use can include taking one or more NSAIDs for three or more (e.g., three, four, five, six, seven, or more) times a week for from about three months to about 36 months (e.g., from about 3 months to about 36 months, from about 3 months to about 32 months, from about 3 months to about 28 months, from about 3 months to about 24 months, from about 3 months to about 22 months, from about 3 months to about 18 months, from about 3 months to about 15 months, from about 3 months to about 12 months, from about 3 months to about 9 months, from about 3 months to about 6 months, from about 6 months to about 36 months, from about 12 months to about 36 months, from about 18 months to about 36 months, from about 24 months to about 36 months, from about 6 months to about 24 months, from about 9 months to about 18 months, from about 12 months to about 15 months, from about 6 months to about 12 months, from about 9 months to about 15 months, from about 12 months to about 18 months, from about 15 months to about 24 months, or from about 18 months to about 28 months). For example, a mammal having membranous nephropathy can have an NSAID-associated membranous nephropathy. Examples of NSAIDs that can be associated with membranous nephropathy identified as having (a) autoantibodies specific for a PCSK6 polypeptide and/or (b) kidney tissue (e.g., the GBM) having an elevated level of a PCSK6 polypeptide include, without limitation, naproxen, ibuprofen, and meloxicam.

Any appropriate method can be used to determine if a mammal (e.g., a human) has autoantibodies specific for a PCSK6 polypeptide. For example, immunological assays using a PCSK6 polypeptide (or a fragment thereof capable of binding to an anti-PCSK6 antibody) can be used to determine if a sample contains autoantibodies specific for a PCSK6 polypeptide. In some cases, an immobilized PCSK6 polypeptide (or an immobilized fragment thereof) can be used to capture an anti-PCSK6 autoantibody if present within a sample being tested, and an anti-Ig antibody (e.g., an anti-human IgG antibody when testing for human autoantibodies) can be used to determine whether or not autoantibodies were captured. In some cases, an anti-Ig antibody can be labeled (e.g., fluorescently or enzymatically labeled) to aid in detection. Any appropriate sample can be used to determine if a mammal (e.g., a human) has autoantibodies specific for a PCSK6 polypeptide. For example, blood samples (e.g., whole blood samples, serum samples, and plasma samples) or urine samples obtained from a mammal being tested can be used to determine if a mammal (e.g., a human) has autoantibodies specific for a PCSK6 polypeptide.

Any appropriate method can be used to determine if a mammal (e.g., a human) has kidney tissue such as the GBM having an elevated level of a PCSK6 polypeptide. For example, immunological techniques such as IHC techniques, immunofluorescence (IF) techniques, or western blot techniques can be used to determine if a mammal (e.g., a human) has kidney tissue such as the GBM having an elevated level of a PCSK6 polypeptide. In some cases, a kidney tissue sample obtained from a mammal to be tested can be stained using an anti-PCSK6 antibody to determine if the mammal has kidney tissue such as the GBM having an elevated level of PCSK6 polypeptides. Any appropriate sample can be used to determine if a mammal (e.g., a human) has kidney tissue such as the GBM having an elevated level of a PCSK6 polypeptide. For example, kidney tissue biopsies can be obtained from a mammal (e.g., a human) being tested and used to determine if the mammal (e.g., the human) has kidney tissue such as the GBM having an elevated level of a PCSK6 polypeptide.

The term “elevated level” as used herein with respect to a PCSK6 polypeptide present within kidney tissue such as the GBM refers to a level of PCSK6 polypeptides present within kidney tissue such as the GBM that is greater (e.g., at least 10, 25, 35, 45, 50, 55, 65, 75, 80, 90, or 100 percent greater) than the median level of PCSK6 polypeptides present within normal kidney tissue (e.g., normal GBM) of comparable mammals not having membranous nephropathy.

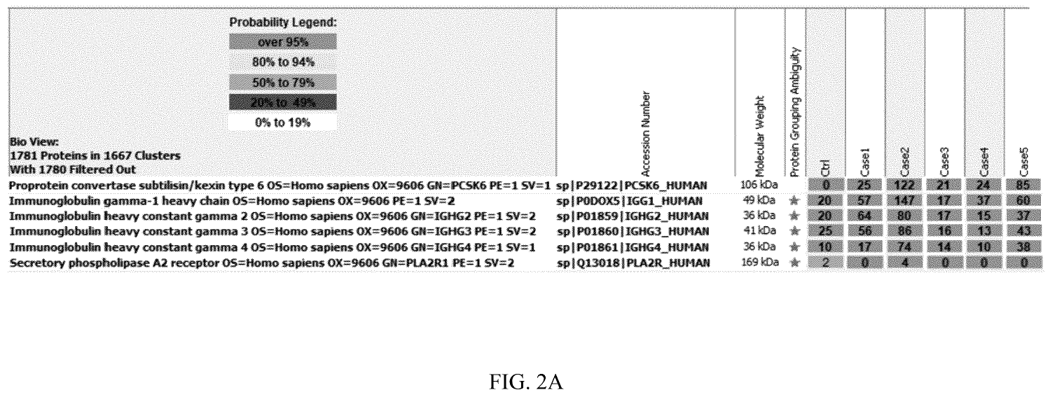

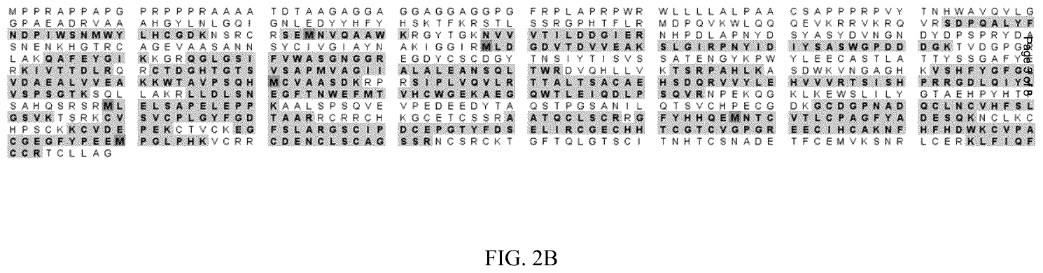

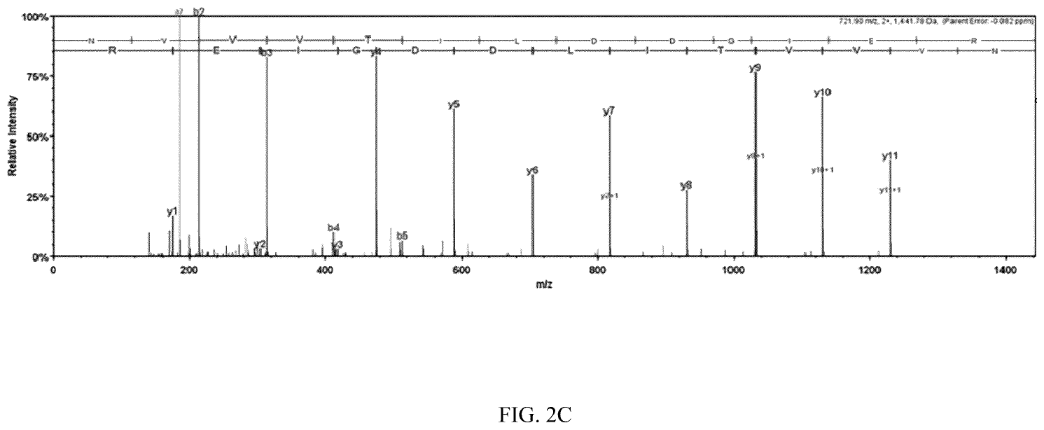

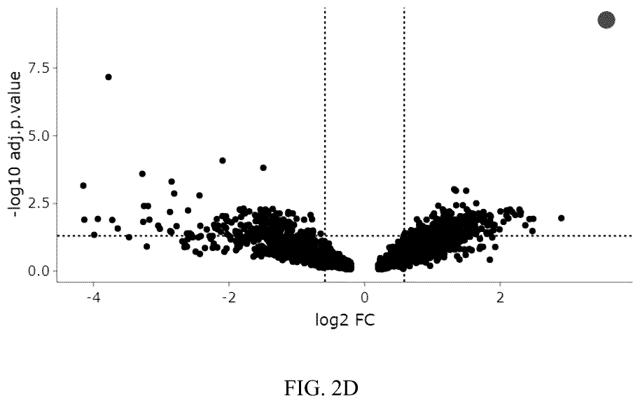

A PCSK6 polypeptide can include any appropriate amino acid sequence. Examples of human PCSK6 polypeptides include, without limitation, those polypeptides having the amino acid sequence set forth in SEQ ID NO: 1 (see, e.g., FIG. 2B) and those polypeptides having the amino acid sequence set forth in The UniProt Knowledgebase (UniProtKB; see, e.g., The UniProt Consortium, Nucleic Acids Research, 51 (D1): D523-D531 (2023)) at accession no. P29122. In some cases, the amino acid sequence of a PCSK6 polypeptide can have a sequence that deviates from the nucleotide sequence set forth in SEQ ID NO:1, sometimes referred to as a variant sequence. For example, a PCSK6 polypeptide can have an amino acid sequence that includes one or more modifications (e.g., deletions, insertions, and substitutions) to the amino acid sequence set forth in SEQ ID NO:1. For example, an amino acid sequence of a PCSK6 polypeptide can have at least 80% sequence identity (e.g., about 82% sequence identity, about 85% sequence identity, about 88% sequence identity, about 90% sequence identity, about 93% sequence identity, about 95% sequence identity, about 97% sequence identity, about 98% sequence identity, or about 99% sequence identity) to the amino acid sequence set forth in SEQ ID NO:1. Percent sequence identity is calculated by determining the number of matched positions in aligned amino acid sequences, dividing the number of matched positions by the length of an aligned amino acid sequence, and multiplying by 100. A matched position refers to a position in which identical amino acids occur at the same position in aligned amino acid sequences. Percent sequence identity also can be determined for any nucleic acid sequence.

The percent sequence identity between a particular nucleic acid or amino acid sequence and a sequence referenced by a particular sequence identification number (e.g., SEQ ID NO:1) is determined as follows. First, an amino acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical amino acid residue is presented in both sequences. The percent sequence identity is determined by dividing the number of matches by the length of the sequence set forth in the identified sequence (e.g., SEQ ID NO:1), followed by multiplying the resulting value by 100. For example, an amino acid sequence that has 340 matches when aligned with the sequence set forth in SEQ ID NO: 1 is 95.5 percent identical to the sequence set forth in SEQ ID NO:1 (i.e., 921÷969×100=95.046). It is noted that the percent sequence identity value is rounded to the nearest tenth. For example, 75.11, 75.12, 75.13, and 75.14 is rounded down to 75.1, while 75.15, 75.16, 75.17, 75.18, and 75.19 is rounded up to 75.2. It also is noted that the length value will always be an integer.

In some cases, a human PCSK6 polypeptide can have the amino acid sequence set forth in FIG. 2B.

Once a mammal (e.g., a human) having membranous nephropathy is identified as having autoantibodies specific for a PCSK6 polypeptide as described herein, the mammal can be classified as having membranous nephropathy that includes the presence of those autoantibodies (e.g., membranous nephropathy that includes the presence of anti-PCSK6 autoantibodies). In some cases, a mammal (e.g., a human) having membranous nephropathy that is identified as having autoantibodies specific for a PCSK6 polypeptide as described herein can be classified as having membranous nephropathy that includes kidney tissue having an elevated level of PCSK6 polypeptides.

Once a mammal (e.g., a human) having membranous nephropathy is identified as having kidney tissue such as the GBM having an elevated level of a PCSK6 polypeptide as described herein, the mammal can be classified as having membranous nephropathy that includes the presence of that kidney tissue (e.g., membranous nephropathy that includes the presence of kidney tissue such as the GBM having an elevated level of PCSK6 polypeptides). In some cases, a mammal (e.g., a human) having membranous nephropathy that is identified as having kidney tissue such as the GBM having an elevated level of a PCSK6 polypeptide as described herein can be classified as having membranous nephropathy that includes autoantibodies specific for a PCSK6 polypeptide.

As described herein, this document also provides methods and materials for treating a mammal having membranous nephropathy. For example, a mammal (e.g., a human) having membranous nephropathy that is identified as having (a) autoantibodies specific for a PCSK6 polypeptide and/or (b) kidney tissue such as the GBM having an elevated level of a PCSK6 polypeptide as described herein can be treated with one or more immunosuppressants. In some cases, a mammal (e.g., a human) having membranous nephropathy that is identified as having (a) autoantibodies specific for a PCSK6 polypeptide and/or (b) kidney tissue such as the GBM having an elevated level of a PCSK6 polypeptide as described herein can be administered, or instructed to self-administer, one or more immunosuppressants to treat membranous nephropathy. In cases where a mammal having membranous nephropathy that is identified as having (a) autoantibodies specific for a PCSK6 polypeptide and/or (b) kidney tissue such as the GBM having an elevated level of a PCSK6 polypeptide as described herein is a chronic NSAID user (e.g., a mammal that previously was administered or that previously was self-administering one or more NSAIDs for extended periods of time), the mammal can stop NSAID use to treat membranous nephropathy. For example, a mammal having membranous nephropathy that previously was administered or that previously was self-administering one or more NSAIDs and that is identified as having (a) autoantibodies specific for a PCSK6 polypeptide and/or (b) kidney tissue such as the GBM having an elevated level of a PCSK6 polypeptide as described herein can stop NSAID use and can be administered, or instructed to self-administer, one or more immunosuppressants to treat membranous nephropathy.

Any appropriate immunosuppressant can be administered to a mammal (e.g., a human that was identified as having (a) autoantibodies specific for a PCSK6 polypeptide and/or (b) kidney tissue such as the GBM having an elevated level of a PCSK6 polypeptide as described herein) to treat membranous nephropathy. In some cases, an immunosuppressant used as described herein to treat membranous nephropathy can reduce inflammation and/or reduce B-cell autoantibody production within a mammal. Examples of immunosuppressants that can be used as described herein to treat membranous nephropathy include, without limitation, mycophenolate mofetil (e.g., Cellcept); steroids such as prednisone; B-cell inhibitors such as anti-CD20 antibodies (e.g., rituximab); calcineurin inhibitors such as cyclosporine and tacrolimus; and mTOR inhibitors such as sirolimus and everolimus.

In some cases, two or more (e.g., two, three, four, five, six, or more) immunosuppressants can be administered to a mammal having membranous nephropathy (e.g., a human that was identified as having (a) autoantibodies specific for a PCSK6 polypeptide and/or (b) kidney tissue such as the GBM having an elevated level of a PCSK6 polypeptide as described herein). For example, two immunosuppressants (e.g., prednisone and Cellcept) can be administered to a human having membranous nephropathy that was identified as described herein.

In some cases, one or more immunosuppressants can be administered to a mammal once or multiple times over a period of time ranging from days to months. In some cases, one or more immunosuppressive drugs can be given to achieve remission of membranous nephropathy, and then given during follow up periods to prevent relapse of the membranous nephropathy.

In some cases, one or more immunosuppressants can be formulated into a pharmaceutically acceptable composition for administration to a mammal (e.g., a human) having membranous nephropathy to reduce inflammation and/or to reduce B-cell autoantibody production within that mammal. For example, a therapeutically effective amount of an immunosuppressant can be formulated together with one or more pharmaceutically acceptable carriers (additives) and/or diluents. A pharmaceutical composition can be formulated for administration in solid or liquid form including, without limitation, in the form of sterile solutions, suspensions, sustained-release formulations, tablets, capsules, pills, powders, or granules.

Pharmaceutically acceptable carriers, fillers, and vehicles that can be used in a pharmaceutical composition described herein can include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

A pharmaceutical composition containing one or more immunosuppressants can be designed for oral or parenteral (including subcutaneous, intramuscular, intravenous, and intradermal) administration. When being administered orally, a pharmaceutical composition can be in the form of a pill, tablet, or capsule. Compositions suitable for parenteral administration can include aqueous and non-aqueous sterile injection solutions that can contain anti-oxidants, buffers, bacteriostats, and solutes that render the formulation isotonic with the blood of the intended recipient. The formulations can be presented in unit-dose or multi-dose containers, for example, sealed ampules and vials, and can be stored in a freeze dried (lyophilized) condition requiring the addition of the sterile liquid carrier, for example, water for injections, immediately prior to use. Extemporaneous injection solutions and suspensions can be prepared from sterile powders, granules, and tablets.

In some cases, a pharmaceutically acceptable composition including one or more immunosuppressants can be administered locally or systemically. For example, a composition provided herein can be administered locally by intravenous injection or blood infusion. In some cases, a composition provided herein can be administered systemically, orally, or by injection to a mammal (e.g., a human).

The effective amount (e.g., effective dose) of a composition containing one or more immunosuppressants can be any amount that reduces inflammation or B-cell autoantibody production (e.g., B-cell antibody production inhibition or reduction in the number of B-cells) within a mammal having membranous nephropathy without producing significant toxicity to the mammal. For example, an effective amount of rituximab to treat membranous nephropathy as described herein can be from about 500 mg to about 1.5 g (e.g., from about 500 mg to about 1.25 g, from about 500 mg to about 1.0 g, from about 500 mg to about 750 mg, from about 750 mg to about 1.5 g, from about 1 g to about 1.5 g, or from about 1.25 g to about 1.5 g) administered IV about two weeks apart. In some cases, an effective amount of rituximab to treat membranous nephropathy as described herein can be from about 200 mg/m² to about 500 mg/m² (e.g., from about 200 mg/m² to about 450 mg/m², from about 200 mg/m² to about 400 mg/m², from about 200 mg/m² to about 375 mg/m², from about 250 mg/m² to about 500 mg/m², from about 300 mg/m² to about 500 mg/m², from about 350 mg/m² to about 500 mg/m², or from about 350 mg/m² to about 400 mg/m²) administered weekly for about four weeks. If a particular mammal fails to respond to a particular amount, then the amount of an immunosuppressant can be increased by, for example, two-fold. After receiving this higher amount, the mammal can be monitored for both responsiveness to the treatment and toxicity symptoms, and adjustments made accordingly. For example, levels of anti-PCSK6 autoantibodies present within the mammal (e.g., within the blood of the mammal) can be monitored by an appropriate method (e.g., ELISA). In some cases, the effective amount of a composition containing one or more immunosuppressants can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition can require an increase or decrease in the actual effective amount administered.

The frequency of administration of one or more immunosuppressants can be any amount that reduces inflammation or B-cell autoantibody production (e.g., B-cell antibody production inhibition or reduction in the number of B-cells) within a mammal having membranous nephropathy without producing significant toxicity to the mammal. For example, the frequency of administration of an immunosuppressant can be from about once a day to about once a month (e.g., from about once a week to about once every other week). The frequency of administration of one or more immunosuppressants can remain constant or can be variable during the duration of treatment. A course of treatment with a composition containing one or more immunosuppressants can include rest periods. For example, a composition containing one or more immunosuppressants can be administered daily over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition may require an increase or decrease in administration frequency.

An effective duration for administering a composition containing one or more immunosuppressants can be any duration that reduces inflammation or B-cell autoantibody production (e.g., B-cell antibody production inhibition or reduction in the number of B-cells) within a mammal having membranous nephropathy without producing significant toxicity to the mammal. In some cases, the effective duration can vary from several days to several months. In general, the effective duration for administering a composition containing one or more immunosuppressants to treat membranous nephropathy can range in duration from about one month to about five years (e.g., from about two months to about five years, from about three months to about five years, from about six months to about five years, from about eight months to about five years, from about one year to about five years, from about one month to about four years, from about one month to about three years, from about one month to about two years, from about six months to about four years, from about six months to about three years, or from about six months to about two years). In some cases, the effective duration for administering a composition containing one or more immunosuppressants to treat membranous nephropathy can be for as long as the mammal is alive. Multiple factors can influence the actual effective duration used for a particular treatment. For example, an effective duration can vary with the frequency of administration, effective amount, use of multiple treatment agents, route of administration, and severity of the condition being treated.

In some cases, a course of treatment and/or the severity of one or more symptoms related to membranous nephropathy can be monitored. Any appropriate method can be used to determine whether or not membranous nephropathy is being treated. For example, immunological techniques (e.g., ELISA) can be performed to determine if the level of anti-PCSK6 autoantibodies present within a mammal being treated as described herein is reduced following the administration of one or more immunosuppressants. Remission and relapse of the disease can be monitored by testing for one or more markers for membranous nephropathy. In some cases, remission can be ascertained by detecting the disappearance or reduction of autoantibodies having the ability to bind to a PCSK6 polypeptide in the sera. In some cases, relapse of membranous nephropathy can be ascertained by a reappearance or elevation of autoantibodies to having the ability to bind to a PCSK6 polypeptide in the sera.

The invention will be further described in the following examples, which do not limit the scope of the invention described in the claims.

### EXAMPLES

### Example 1: Proprotein Convertase Subtilisin Kexin Type 6 (PCSK6) is the Likely Antigenic Target in Membranous Nephropathy and NSAID Use

This Example identifies PCSK6 as a target antigen in NSAID-associated membranous nephropathy, and describes how PCSK6 can be used as a serological biomarker and/or therapeutic target of NSAID-associated membranous nephropathy.

### Methods

### Discovery Cohort (Mayo Clinic)

### Patients and Sample Collection (Discovery Cohort)

Biopsies received in the Renal Pathology Laboratory, Department of Laboratory Medicine and Pathology, Mayo Clinic, for diagnosis and interpretation were evaluated. The diagnosis of membranous nephropathy (MN) was confirmed by light microscopy (LM), immunofluorescence (IF) microscopy including PLA2R studies, and electron microscopy (EM). The clinical information was obtained from the accompanying charts. For detection of novel proteins, MS/MS was performed in 250 cases of PLA2R-negative cases (discovery cohort) that included cases used for identification of EXT1/EXT2, NELL1, SEMA3B, PCDH7, FAT1, and NDNF (Sethi et al., J. Am. Soc. Nephrol., 30:1123-1136 (2019); Sethi et al., Kidney International, 97:163-174 (2020); Sethi et al., Kidney International, 98:1253-1265 (2020); Sethi et al., J. Am. Soc. Nephrol., 32:1249-1261 (2021); and Sethi et al., J. Am. Soc. Nephrol., 33:1033-1044 (2022)). A novel protein, PCSK6, was detected in 5 cases of PLA2R-negative MN (PCSK6-associated MN) by MS/MS. All five cases were negative for spectral counts for THSD7A, EXT1/EXT2, NELL1, SEMA3B, HTRA1, CNTN1, NCAM1, PCDH7, and FAT1 while baseline spectral counts for PLA2R was present in 1 of the 5 cases.

For control cases, MS/MS was performed on 116 cases that included 15 cases of time zero kidney transplant biopsies, 17 cases of minimal change disease, 44 cases of focal segmental glomerulosclerosis, 7 cases of diabetic glomerulosclerosis, 5 cases of IgA nephropathy, and 28 cases of PLA2R-associated MN. None of the control cases showed any spectral counts for PCSK6. The initial 250 PLA2R-negative MN (discovery cohort) and control cases have been characterized and previously utilized for the discovery of EXT1/EXT2, NELL1, SEMA3B, PCDH7, FAT1, and NDNF (Sethi et al., J. Am. Soc. Nephrol., 30:1123-1136 (2019); Sethi et al., Kidney International, 97:163-174 (2020); Sethi et al., Kidney International, 98:1253-1265 (2020); Sethi et al., J. Am. Soc. Nephrol., 32:1249-1261 (2021); and Sethi et al., J. Am. Soc. Nephrol., 33:1033-1044 (2022)).

### Protein Identification by Laser Capture Microdissection, Trypsin Digestion, Nano-LC Orbitrap Tandem Mass Spectrometry (MS/MS) (Discovery Cohort)

For each case, 10-micron thick formalin-fixed paraffin sections (FFPE) were obtained and mounted on a special PEN membrane laser microdissection slide and using a Zeiss Palm Microbean microscope, the glomeruli were microdissected to reach approximately 250-550000 μM² per case. Resulting FFPE fragments were digested with trypsin and collected for MS/MS analysis. The trypsin digested peptides were identified by nano-flow liquid chromatography electrospray tandem MS/MS (nanoLC-ESI-MS/MS) using a Thermo Scientific Q-Exactive or Exploris480 Mass Spectrometer (Thermo Fisher Scientific, Bremen, Germany) coupled to a Thermo Ultimate 3000 RSLCnano HPLC system. All MS/MS samples were analyzed using Mascot and X! Tandem set up to search a Swissprot human database. Scaffold (version 4.8.3, Proteome Software Inc., Portland, OR) was used to validate MS/MS based peptide and protein identifications. Peptide identifications were accepted at greater than 95.0% probability by the Scaffold Local FDR algorithm with protein identifications requiring a 2-peptide minimum and a 95% probability using Protein Prophet.

The glomerular areas dissected for each PCSK6-associated MN was as follows: case 1=85952 μM², case 2=534119 μM², case 3=537081 μM², case 4=693343 μM², and case 5=659849 μM².

### Immunohistochemical (IHC) Staining for PCSK6 (Discovery Cohort)

IHC staining was performed using the Leica Bond RX stainer (Leica). FFPE tissues were sectioned at 5 microns and IHC staining was performed on-line. Slides for PCSK6 stain were pretreated for 5 minutes using Enzyme 1 (AR9551; Leica) and incubated in Protein Block (Dako) for 5 minutes. The PCSK6 primary antibody (Rabbit Polyclonal; Novus, catalog #NBP1-87354) was diluted to 1:50 in Background Reducing Diluent (Dako) and incubated for 30 minutes.

The detection system used was Polymer Refine Detection System (Leica). This system includes the hydrogen peroxidase block, post primary and polymer reagent, DAB, and hematoxylin. Immunostaining visualization was achieved by incubating slides 10 minutes in DAB and DAB buffer (1:19 mixture) from the Bond Polymer Refine Detection System. To this point, slides were rinsed between steps with 1× Bond Wash Buffer (Leica). Slides were counterstained for five minutes using Schmidt hematoxylin and molecular biology grade water (1:1 mixture), followed by several rinses in 1× Bond wash buffer and distilled water, this was not the hematoxylin provided with the Refine kit. Once the immunochemistry process was completed, slides were removed from the stainer and rinsed in tap water for five minutes. Slides were dehydrated in increasing concentrations of ethyl alcohol and cleared in 3 changes of xylene prior to permanent cover-slipping in xylene-based medium.

Elution of IgG from Kidney Biopsy Specimens (Discovery Cohort)

IgG was acid eluted from frozen (tissue remaining after immunofluorescence microscopy) kidney biopsy specimens. The eluate containing anti-PCSK6F IgG was obtained from the 2 patients with PCSK6-associated MN that were pooled together. The eluate containing control IgG was obtained from 2 patients with PLA2R-associated MN. Serial cryostat sections of 4 μm thickness were mounted side by side on a glass slide. The slides were thawed, fixed with pre-chilled 100% acetone for 10 minutes at room temperature and then washed for 5 minutes with phosphate-buffered saline (0.01 M, phosphate, pH 7.2). The slide sections were covered with 0.2 mL of a 0.02 M citrate buffer (pH 3.2) and incubated overnight in a humid chamber at 4° C. The eluate was extracted with a calibrated syringe and neutralized with 0.4 M NaOH to a pH of 7.2.

### Western Blot Analysis (Discovery Cohort)

A recombinant protein corresponding to antigenic determinants in human PCSK6 (Abnova©, Taipei, Taipei city, Taiwan) was used under non-reducing and reducing conditions. The target molecular weight and dominant band is expected at 37 kDa. The protein (1600 ng) was diluted with reduced (with β-mercaptoethanol) Laemmli sample buffer (BioRad) and boiled for 5 minutes. The samples were loaded into Criterion 12% TGX gels (BioRad) and electrophoresed in Tris-glycine-SDS running buffer. Proteins were transferred to nitrocellulose membrane (0.20 μm pore size) according to standard protocols. Membranes were incubated overnight at 4° C. with mouse anti-human PCSK6 (1:5000, recommended dilution Antibodies online), eluates from PCSK6-associated MN and PLA2R-associated MN, patient serum (1:50) from PCSK6 associated MN at baseline and after treatment with RTX, patient serum (1:50) from PLA2R associated MN, and patient serum (1:50) from MPO-AAV-GN. Subsequently, blots were washed and incubated 1 hour at room temperature with a secondary anti-mouse IgG Fc (1:15000) and anti-human IgG Fc (1:5000). Near infrared fluorescence was detected at the 700 and 800 nm channels in the Odyssey Infrared Imaging System (LI-CORR Biosciences, Lincoln, NE). The reading was done for 10 minutes in both channels (total 30 minutes).

### Validation Cohort (Arkana Laboratories)

The validation cohort cases were obtained from the clinical case archives at Arkana Laboratories. Kidney biopsies were chosen with the following inclusion criteria 1) diagnosis of membranous glomerulopathy or membranous lupus nephritis (ISN/RPS class V, without concurrent proliferative lupus nephritis), 2) adequacy of ≥10 glomeruli on FFPE tissue and ≥ 5 glomeruli within frozen tissue, and 3)≤50% interstitial fibrosis and tubular atrophy. Cases that were PLA2R-positive (n=96), THSD7A-positive (n=60), EXT1/2-positive (n=52), and NELL1-positive (n=57) were included as controls for known antigen type for specificity. Biopsies negative for all four antigens were used for discovery (n=118).

### Protein G Immunoprecipitation (Validation Cohort)

Immunoprecipitation with protein G was performed to recover immune complexes from residual frozen kidney biopsy tissue. Briefly, biopsy cores were washed 3 times in phosphate buffered saline and were then dissociated into a protein lysate through bead-beating in 250 μL Pierce lysis buffer (Thermo-Fisher, catalog #87787) containing 1% Halt™ protease inhibitor (Thermo-Fisher, catalog #78440). 50 μL protein G magnetic beads (Thermo-Fisher, catalog #1003D) were added to the supernatant, which was incubated at room temperature for 1 hour with shaking. Beads were washed in PBS, followed by digestion with trypsin for MS preparation.

### Mass Spectrometry of Protein G Immunoprecipitates (Validation Cohort)

Peptides were produced through trypsin digestion of the immune complexes adhered to protein G beads post-immunoprecipitation. DIA mass spectrometry was used for analysis as follows. An UltiMate 3000 RSLC nano system (Thermo Scientific) was used to separate tryptic peptides on a 150×0.075 mm column packed with a reverse phase XSelect CSH C18 2.5 μm resin. The peptides were eluted with a gradient of buffer A (0.1% formic acid+0.5% acetonitrile)/buffer B (0.1% formic acid+99.9% acetonitrile), from a 97:3 ratio to a 60:40 ratio over 1 hour. Electrospray (2.25 kV) was used to ionize the eluted peptides on an Orbitrap Exploris 480 mass spectrometer (Thermo Scientific). Six gas-phase fractions of a pooled sample were acquired on the Orbitrap Exploris mass spectrometer with 4 m/z DIA spectra (30,000 resolution, normalized AGC target 100%, maximum inject time 66 ms) in a staggered window pattern to assemble a chromatogram library. Precursor spectra were acquired after each DIA duty cycle, spanning the m/z range of the gas-phase fraction (i.e., 496-602 m/z, 596-702 m/z). For wide-window acquisitions, precursor spectra from each DIA cycle were acquired (385-1015 m/z, 60,000 resolution, normalized AGC target 100%, maximum injection time 50 ms), followed by 50×12 m/z DIA spectra (12 m/z precursor isolation windows at 15,000 resolution, normalized AGC target 100%, maximum injection time 33 ms) using a staggered window pattern with optimized window placements.

ScaffoldDIA (Proteome software) was used to configure DIA library searches. Narrow-window DIA files were searched against the human predicted spectral library from Prosit (2019/04 Uniprot) with 10 ppm precursor and fragment ion tolerances to generate an empirically corrected chromatogram library with a peptide and protein false discovery rate of 1%. Wide-window DIA files were subsequently searched against the generated empirically corrected chromatogram library.

### Confocal Microscopy (Validation Cohort)

For confocal microscopy, tissue sections were co-stained with PCSK6 and IgG by paraffin immunofluorescence as follows. 4 μm FFPE tissue sections were deparaffinized, followed by antigen retrieval with Proteinase K. Sections were incubated with PCSK6 antibody (Novus Biologicals, cat #NBP1-87354) at 1:50 dilution for 30 minutes at room temperature. Tissue sections were washed with phosphate buffered saline and Rhodamine Red™-X AffiniPure goat anti-rabbit IgG (Jackson Immunoresearch, cat #111-295-144) was applied as a secondary antibody at 1:100 dilution for 30 minutes. Tissue sections were then co-stained by adding FITC-conjugated polyclonal anti-human IgG (Agilent) at 1:40 dilution for 30 minutes. Slides were cover-slipped in anti-fade mounting medium and imaged under a Leica SBA DMI8 confocal laser scanning microscope. PLA2R-positive MN cases were co-stained in parallel to cases of interest as negative controls.

### Results

### Discovery Cohort

### MS/MS Detection of PCSK6 in PLA2R-Negative MN Biopsies in the Discovery Cohort

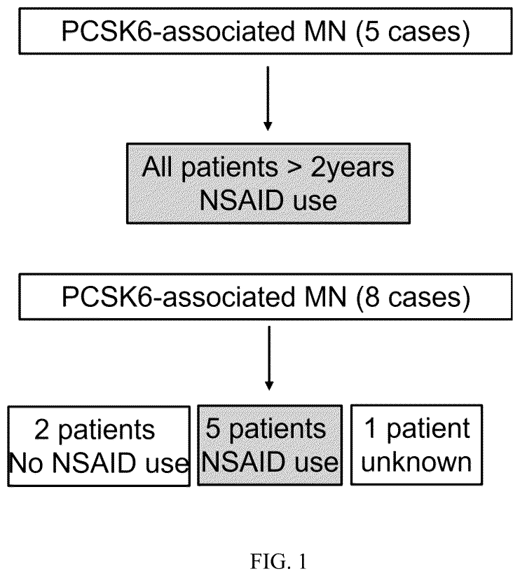

A unique protein, PCSK6, was detected by MS/MS in the glomeruli of 5 (2%) cases of MN from the 250 PLA2R-negative cases of the discovery cohort cases (FIG. 1). The total spectral counts of PCSK6 ranged from 21-122 in the 5 cases with an average total spectral count of 55.4 (SD±45.8). All control cases including 15-time zero transplant biopsies, 73 other glomerulopathies, and 28 PLA2R-associated MN cases were negative for PCSK6. The total spectral counts of five cases of PCSK6-positive MN along with a representative sequence coverage map of PCSK6 are shown in FIG. 2A-B. The MS/MS spectra match from one case is shown in FIG. 2C. None of the PCSK6-positive cases show any spectral counts for EXT1/EXT2, THSD7A, NELL1, NCAM1, CNTN1, HTRA1, SEMA3B, PCDH7, FAT1, and NDNF while baseline PLA2R counts were detected in one of the 5 cases.

### Immunohistochemical Studies (IHC)

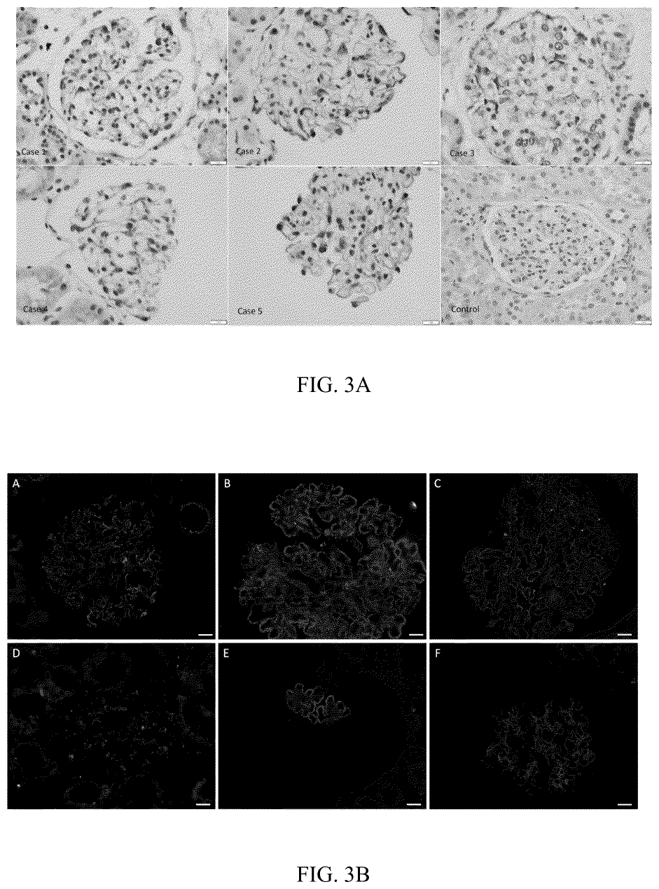

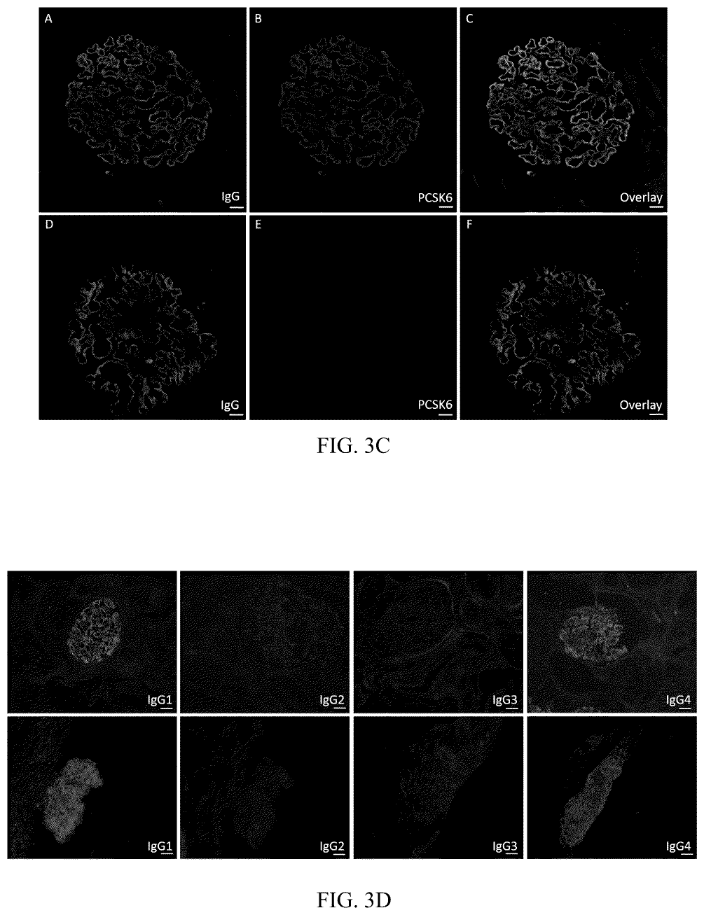

IHC staining was performed on biopsy samples in all 5 cases of the discovery cohort and showed granular staining (2-3+) along the GBM in all cases (FIG. 3A). Segmental staining was present in case 1. A control PLA2R-associated MN is shown that was negative for PCSK6 staining along the GBM.

### IgG Elution and Western Blot Studies

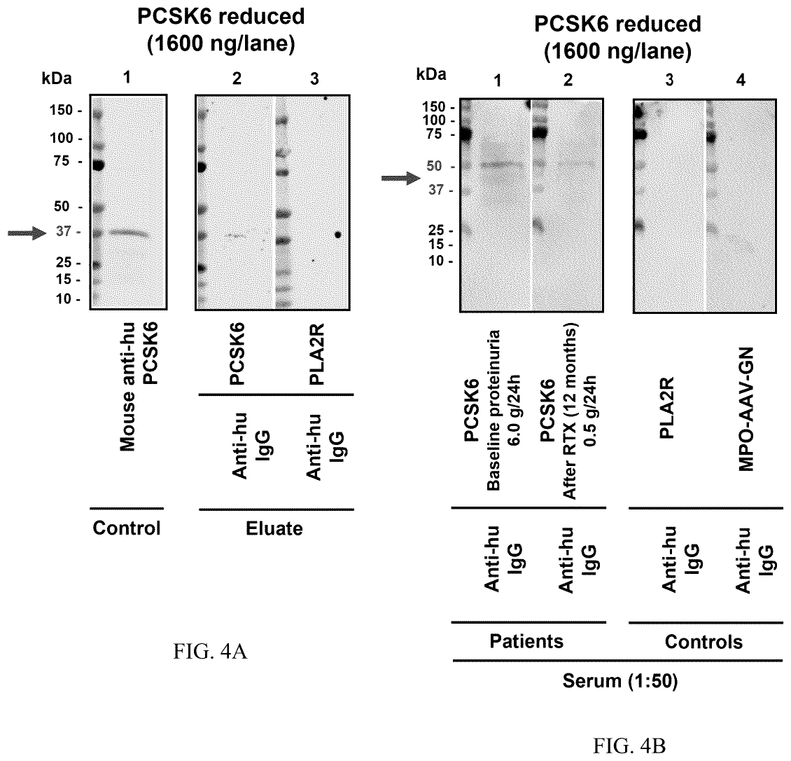

Western blot analyses were performed using recombinant human PCSK6 to determine the presence of anti-PCSK6 antibodies in the eluate obtained from 2 pooled kidney biopsies of PCSK6-associated MN cases (FIG. 4A). PCSK6 was detected by mouse anti-human PCSK6 under reducing conditions. FIG. 4A shows a dominant band at approximately 37 kDa to reduced PCSK6 by mouse anti-human PCSK6 (lane 1, positive control). The same band was detected after exposure of reduced human PCSK6 to IgG obtained from the eluate of PCSK6-associated MN (lane 2). Anti-PCSK6 antibodies were not detected in IgG eluate obtained from patients with PLA2R-associated-MN (lane 3). In the serum studies, PCSK6 was detected by anti-PCSK6 IgG from patient serum under reducing conditions (FIG. 4B). Binding of anti-PCSK6 IgG to a band between 37-50 kDa was detected after exposure of reduced human PCSK6 to serum from patients with PCSK6-associated MN at baseline (lane 1) with a decrease of the amount of antibodies bound in the serum after treatment with rituximab and decrease in proteinuria (lane 2) (FIG. 4B). Anti-PCSK6 antibodies were not detected in serum obtained from patients with PLA2R-associated-MN and MPO-AAV-GN (lane 3 and 4).

### Validation Cohort

### MS/MS Detection of PCSK6 in PLA2R-Negative MN Biopsies in the Validation Cohort

MS was performed on 118 cases of PLA2R-/THSD7A-/NELL1-/EXT2-quadruple negative MN/MLN biopsies from the validation cohort following protein G immunoprecipitation to elute antibodies from frozen biopsy tissue. PSCK6 was independently detected as a uniquely enriched protein in 6 of the cases (FIG. 1 and FIG. 2D). The frequency of this protein target could not be determined within this MS cohort, as it was enriched for patients with a history of autoimmune disease. PCSK6 was not identified in PLA2R (n=96), THSD7A (n=60), NELL1 (n=57), or EXT2-positive (n=52) control cases. PCSK6 was confirmed as a protein target in two of the six cases by performing MS on paired FFPE tissue following laser capture microdissection. The remaining 4 cases were confirmed by immunostaining on paraffin immunofluorescence. In addition to the MS cases, PCSK6 was identified as a target antigen in two additional patients with MN in the setting of high dose NSAID use by immunofluorescence microscopy.

### Immunofluorescence and Confocal Microscopy

Both paraffin immunofluorescence and confocal microscopy were performed in the validation cohort to show staining for PCSK6 and colocalization of IgG and PCSK6 along the GBM. Immunofluorescence microscopy showed granular staining of PCSK6 along the GBM in all cases (FIG. 3B), and confocal microscopy showed colocalization of IgG and PCSK6 along the GBM (FIG. 3C). Control PLA2R-associated MN was negative for PCSK6.

### Clinical and Laboratory Findings of PCSK6-Associated MN (Combined Discovery and Validation Cohorts, Table 1)

The mean age of PCSK6-associated MN was 46.9±11.8 years (range 32-66, median 50). There were 7 males and 6 females. Eight patients were white, one black, one Alaskan native, one American Indian, and in 2 cases the race was not known. The mean serum creatinine and proteinuria at kidney biopsy was 0.93±0.47 mg/dl and 6.5±3.3 g/day, respectively. All five cases of the discovery cohort had history of prolonged use (>2 years) of NSAID use. These included naproxen, ibuprofen, and meloxicam. In the validation cohort, five cases had history of NSAID use, 2 had no history of NSAID use, and in 1 case history was not available. Taken together 10 (83.3%) out 12 patients had history of NSAID use. In addition, of the 8 cases of the validation cohort, 1 case had Hashimoto's thyroiditis, two had Sjogren's syndrome, one had lupus, and 4 had no associated autoimmune disease. In the discovery cohort, one patient had rheumatoid arthritis and the remaining patients had no associated disease.

Kidney biopsy revealed a MN in all cases with thickened glomerular basement membranes and no proliferative findings. There was no or minimal (≤10%) tubular atrophy and interstitial fibrosis in 12 of 13 patients (92.3%). Immunofluorescence findings revealed granular IgG (2-3+) and C3 (1-3+) along the GBM in all cases. There was no tubular basement membrane staining for IgG in any of the cases. Electron microscopy revealed subepithelial electron dense deposits in all cases, in most cases GBM extension between deposits was present (stage II MN of Ehrenreich and Churg). Subendothelial deposits were absent. Sparse mesangial deposits were present in a small subset of patients. PLA2R staining was negative in all cases. IgG subclass staining of three cases from the validation cohort showed IgG1 and IgG4 co-dominance (3+ each) (FIG. 3D).

Follow-up was available for all patients (Table 1). Eight (61.5%) patients achieved complete remission, three (23.1%) achieved partial remission, while 2 (15.4%) patients continued to have significant proteinuria and 1 patient had a relapse. One (patient 12) of the 2 patients that continued to have significant proteinuria was recent case with a follow-up of only 2 months. NSAIDs were discontinued in all patients in the discovery cohort and in all patients in the validation cohort that had history of NSAID use. Seven patients were treated conservatively, three received rituximab, one received cyclosporine, one received mycophenolate mofetil (did not achieve remission), and one initially received rituximab followed by cyclophosphamide when the patient had a relapse. Patient 1 and 2 received rituximab as part of a clinical trial. Follow up for the discovery cohort was from 26 months to 128 months, and follow-up of the validation cohort was from 2 months to 40 months.

TABLE 1
Clinical and pathology findings. Patients 1-5 are from the discovery cohort, and patients 6-13 are from the validation cohort.

| | | | Urinary | | | | | | | | |
|---|---|---|---|---|---|---|---|---|---|---|---|
| | | Serum | Protein | | | | Remission/ | Total/ | | | |
| | Age/ | Cr | (g/24 | NSAID | Autoimmune | | Last follow | sclerosed | IFTA | | EM |
| Case | Sex | mg/dL | hours) | use | disease | Treatment | up in months* | glomeruli | % | IF | stage |
| 1 | 50/F | 0.6 | 9.2 | Yes | Rheumatoid | Rituximab | Complete | 17/0 | 0 | IgG 3+ | II |
| | | | | | arthritis | | remission/60 | | | IgM 1+ | |
| | | | | | | | | | | C3 2+ | |
| 2 | 54/M | 1.2 | 7.2 | Yes | no | Rituximab | Complete | 16/0 | 0 | IgG 3+ | I-II |
| | | | | | | | remission/128 | | | C3 2+ | Few mes |
| 3 | 55/M | 0.9 | 12.4 | Yes | no | Conservative | Complete | 12/1 | 0 | IgG 3+ | II |
| | | | | | | | remission/26 | | | C3 1+ | |
| 4 | 53/M | 0.9 | 7.0 | Yes | Arthralgias, ANA | Steroids, | Complete | 14/0 | 0 | IgG 3+ | II |
| | | | | | 1:80, negative | cyclosporine | remission/115 | | | IgA 1+ | Mes |
| | | | | | dsDNA | | | | | C3 2+ | |
| 5 | 61/F | 0.9 | 3.6 | Yes | no | Conservative | Partial | 12/1 | 0 | IgG 3+ | II |
| | | | | | | | remission/11 | | | IgA 1+ | |
| | | | | | | | | | | C3 1+ | |
| 6 | 34/M | 1 | 5.0 | Yes | no | Conservative | Complete | 37/0 | 0 | IgG3+ | I-II |
| | | | | | | | remission/37 | | | C3 1+ | |
| 7 | 58/F | 0.6 | 8.3 | No | Sjogren's | Conservative | Partial | 80/5 | 0 | IgG 3+ | I |
| | | | | | | | remission/20 | | | C3 trace | Mes |
| 8 | 40/F | 0.6 | 10.1 | Yes | Sjogren's | Conservative | Complete | 57/0 | 0 | IgG 3+ | II |
| | | | | | | | remission/24 | | | IgA 1+ | |
| | | | | | | | | | | C3 2+ | |
| 9 | 37/M | 2.26 | 1.9 | No | SLE | Mycophenolate | No | 54/7 | 30 | IgG 3+ | III |
| | | | | | | mofetil | remission/40 | | | IgM 2+ | Mes |
| | | | | | | | | | | C3 3+ | |
| 10 | 38/M | 0.76 | 1.0 | Unknown | no | Rituximab | Partial | 90/6 | 10 | IgG 3+ | I-II |
| | | | | | | followed by | remission/24 | | | C3 1+ | Mes |
| | | | | | | cyclophosphamide | | | | | |
| 11 | 32/F | 0.4 | 5.7 | No | Hashimoto's | Rituximab | Complete | 43/2 | 5 | IgG 3+ | I-II |
| | | | | | thyroiditis | | remission/25 | | | IgA trace | |
| | | | | | | | | | | C3 1+ | |
| 12 | 32/M | 1.28 | 2.53 | Yes | No | Conservative | No | 20/0 | 10 | IgG 3+ | III |
| | | | | | | | remission/2 | | | C3 3+ | |
| 13 | 66/F | 0.7 | 6.5 | Yes | Hypothyroidism | Conservative | Complete | 41/3 | 0 | IgG 3+ | I |
| | | | | | | | remission/37 | | | | |

*Clinical end points were defined as: Complete remission, proteinuria <0.3 g/d; partial remission, proteinuria <3.5 g/d and a <50% reduction from baseline proteinuria; and no remission, <50% reduction from baseline proteinuria.

Together, these results demonstrate that the PCSK6 polypeptide is the target antigen in a NSAID-associated membranous nephropathy. Accordingly, the presence of (a) autoantibodies specific for a PCSK6 polypeptide and/or (b) a kidney tissue such as the GBM having an elevated level of a PCSK6 polypeptide can be used to identify a mammal as having a PCSK6-positive membranous nephropathy. In some cases, a mammal (e.g., a human that uses NSAIDs chronically) identified as having a PCSK6-positive membranous nephropathy can be treated by administering one or more immunosuppressive agents to the mammal.

### Example 2: Identifying PCSK6 Polypeptide Positive Membranous Nephropathy

A blood sample (e.g., serum) is obtained from a human having membranous nephropathy. The obtained sample is examined for the presence of autoantibodies specific for a PCSK6 polypeptide.

If autoantibodies specific for a PCSK6 polypeptide are detected in the sample, as compared to a control level, then the human is classified as having a PCSK6 positive membranous nephropathy.

### Example 3: Treating PCSK6 Polypeptide Positive Membranous Nephropathy

A human identified as having autoantibodies specific for a PCSK6 polypeptide is administered one or more immunosuppressive agents (e.g., one or more corticosteroids, cyclosporine, and/or one or more B-cell reduction or depletion agents such as rituximab).

The administered immunosuppressive agent(s) can reduce inflammation and/or B-cell autoantibody production.

The administered immunosuppressive agent(s) can reduce the level of autoantibodies specific for a PCSK6 polypeptide present within the human.

### Example 4: Treating PCSK6 Polypeptide Positive Membranous Nephropathy

A human that is a chronic NSAID user (e.g., a mammal that previously was administered or that previously was self-administering one or more NSAIDs for extended periods of time) and is identified as having autoantibodies specific for a PCSK6 polypeptide stops NSAID use and is administered one or more immunosuppressive agents (e.g., one or more corticosteroids, cyclosporine, and/or one or more B-cell reduction or depletion agents such as rituximab).

The administered immunosuppressive agent(s) can reduce inflammation and/or B-cell autoantibody production.

The administered immunosuppressive agent(s) can reduce the level of autoantibodies specific for a PCSK6 polypeptide present within the human.

### Example 5: Identifying PCSK6 Polypeptide Positive Membranous Nephropathy

A kidney tissue sample is obtained from a human having membranous nephropathy. The obtained sample is examined for an elevated level of a PCSK6 polypeptide.

If an elevated level of a PCSK6 polypeptide is detected in the sample, as compared to a control level, then the human is classified as having a PCSK6 polypeptide positive membranous nephropathy.

### Example 6: Treating PCSK6 Polypeptide Positive Membranous Nephropathy

A human identified as having an elevated level of a PCSK6 polypeptide in kidney tissue such as the GBM is administered one or more immunosuppressive agents (e.g., one or more corticosteroids, cyclosporine, and/or one or more B-cell reduction or depletion agents such as rituximab).

The administered immunosuppressive agent(s) can reduce inflammation and/or B-cell autoantibody production.

The administered immunosuppressive agent(s) can reduce a level of autoantibodies specific for a PCSK6 polypeptide present within the human.

### Example 7: Treating PCSK6 Polypeptide Positive Membranous Nephropathy

A human that is a chronic NSAID user (e.g., a mammal that previously was administered or that previously was self-administering one or more NSAIDs for extended periods of time) and is identified as having an elevated level of a PCSK6 polypeptide in kidney tissue such as the GBM stops NSAID use and is administered one or more immunosuppressive agents (e.g., one or more corticosteroids, cyclosporine, and/or one or more B-cell reduction or depletion agents such as rituximab).

The administered immunosuppressive agent(s) can reduce inflammation and/or B-cell autoantibody production.

The administered immunosuppressive agent(s) can reduce the level of autoantibodies specific for a PCSK6 polypeptide present within the human.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating membranous nephropathy, wherein said method comprises:
(a) identifying a mammal as having membranous nephropathy comprising (i) autoantibodies specific for a PCSK6 polypeptide, (ii) kidney tissue comprising an elevated level of said PCSK6 polypeptide, or both (i) and (ii), and
(b) administering an immunosuppressant to said mammal.

2. The method of claim 1, wherein said mammal is a human.

3. The method of claim 1, wherein said mammal is a mammal that was previously administered a NSAID.

4. The method of claim 1, wherein said immunosuppressant is a B-cell inhibitor, a calcineurin inhibitor, or a mTOR inhibitor.

5. The method of claim 1, wherein said immunosuppressant is selected from the group consisting of B rituximab, cyclosporine, tacrolimus, and everolimus.

6. The method of claim 1, wherein a level of autoantibodies present within said mammal is reduced by at least 5 percent following said administering step.

7. A method for treating membranous nephropathy, wherein said method comprises administering an immunosuppressant to a mammal identified as having membranous nephropathy comprising (i) autoantibodies specific for a PCSK6 polypeptide, (ii) kidney tissue comprising an elevated level of said PCSK6 polypeptide, or both (i) and (ii).

8. The method of claim 7, wherein said mammal is a human.

9. The method of claim 7, wherein said mammal is a mammal that was previously administered a NSAID.

10. The method of claim 7, wherein said immunosuppressant is a B-cell inhibitor, a calcineurin inhibitor, or a mTOR inhibitor.

11. The method of claim 7, wherein said immunosuppressant is selected from the group consisting of rituximab, cyclosporine, tacrolimus, and everolimus.

12. The method of claim 7, wherein a level of autoantibodies present within said mammal is reduced by at least 5 percent following said administering step.

13. A method for treating a mammal having membranous nephropathy and kidney tissue comprising an elevated level of a PCSK6 polypeptide, wherein said method comprises administering an immunosuppressant to said mammal.

14. The method of claim 13, wherein said mammal is a human.

15. The method of claim 13, wherein said mammal is a mammal that was previously administered a NSAID.

16. The method of claim 13, wherein said mammal comprises autoantibodies specific for said PCSK6 polypeptide.

17. The method of claim 13, wherein said mammal was identified as having said kidney tissue.

18. The method of claim 13, wherein said immunosuppressant is a B-cell inhibitor, a calcineurin inhibitor, or a mTOR inhibitor.

19. The method of claim 13, wherein said B immunosuppressant is selected from the group consisting of rituximab, cyclosporine, tacrolimus, and everolimus.

20. The method of claim 13, wherein a level of autoantibodies present within said mammal is reduced by at least 5 percent following said administering step.
